(19) United States
(12) Patent Application Publication
(10) Pub. No.: US 2016/0073895 A1
(43) Pub. Date: Mar. 17, 2016

(54) System and Method for a Physical Training Test Facilitating System

(71) Applicant: Derek A. Daly, Miami, FL (US)

(72) Inventor: Derek A. Daly, Miami, FL (US)

(21) Appl. No.: 14/681,498

(22) Filed: Apr. 8, 2015

Related U.S. Application Data
(60) Provisional application No. 62/049,213, filed on Sep. 11, 2014.

(51) Int. Cl. A61B 5/01; G09B 19/00

(52) U.S. Cl. CPC A61B 5/01; G09B 19/00

(57) ABSTRACT

A physical training test facilitating system utilizes a plurality of subject evaluation sensors in conjunction with a processing unit, body recognition software, exercise recognition software and exercise evaluation software to administer and produce an objective score for a physical training test. The sensors capture test subject data while the test subject is performing exercise routines, which is processed in order to recognize the exercise routines and evaluate them against exercise form evaluation standards.

The current application claims a priority to the U.S. Provisional Patent application Ser. No. 62/049,213 filed on Sep. 11, 2014.

### FIELD OF THE INVENTION

The present invention relates generally to physical fitness tests. More specifically, the present invention relates to administering and objectively measuring physical fitness tests.

### BACKGROUND OF THE INVENTION

Currently, the physical fitness test in the military is administered by other servicemen. For example, the push-up and sit-up events are currently graded by the naked eye.

It is therefore an objective of the present invention to provide a more objective means of evaluating the performance of users in physical fitness exams, with elements of computer automation and standardization. The invention will accurately streamline every physical training event, by having the evaluation be computer-based and more objective, making it fair for everyone involved. A soldier would not be able to cheat or negatively influence the events. This solution will also eliminate the errors associated with grading with the human eye. Soldiers will not be able to cheat the test by knowing or influencing the graders. This system will also motivate the younger generations to improve their own physical fitness by introducing virtual training.

In the future one can foresee soldiers, sailors, and airmen ultimately purchasing this technology (maybe tied to their video game console) for their own homes. They will be able to track their history and current event score and even view themselves in action in the comfort of their home. This will keep them knowledgeable of their scores and techniques and motivate them to get better. In alternative or future embodiments of the invention, the invention's system will be able to measure body fat composition or track and grade other events. This type of technology could be used in civilian gyms as a personal trainer or to test and track progression in physical training.

### BRIEF DESCRIPTION OF THE DRAWINGS

FIG. 1 is a schematic diagram of the system of the present invention.

FIG. 2 is a stepwise flow diagram describing the general method of the present invention.

FIG. 3 is a stepwise flow diagram describing steps for producing a PT test score.

FIG. 4 is a stepwise flow diagram describing steps for evaluating exercise routines.

FIG. 5 is a stepwise flow diagram describing steps for initializing and administering the PT test.

FIG. 6 is a stepwise flow diagram describing steps for evaluating a running exercise routine.

FIG. 7 is a stepwise flow diagram describing steps for monitoring a test subject's vital signs.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced without the implementation of some features as they are described. It should be understood that some details have not been described in detail in order to not unnecessarily obscure focus of the invention.

The present invention is a system and method for facilitating a physical training (PT) test by objectively evaluating test subject performance in order to curtail incidents of cheating.

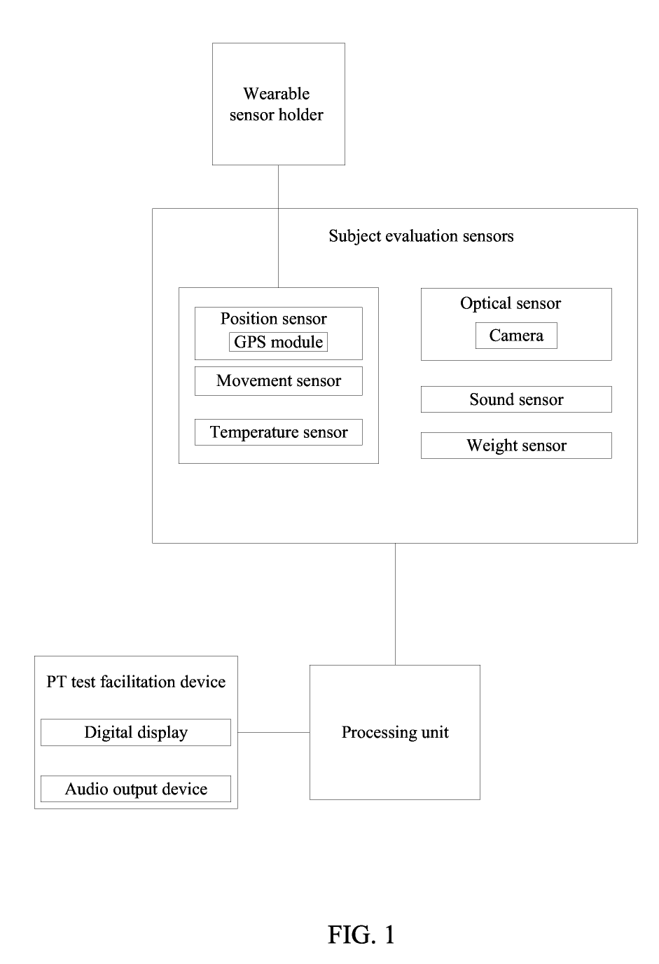

Referring to FIG. 1, the system of the present invention generally comprises a wearable sensor holder, a plurality of subject evaluation sensors, and a processing unit. The wearable sensor holder is intended to be worn by a test subject during a PT test in order to facilitate tracking and evaluation of exercise routines. Each of the plurality of subject evaluation sensors is electronically connected to the processing unit, wherein the processing unit receives inputs from the plurality of subject evaluation sensors in order to facilitate a PT test through a PT test facilitation device. Additionally, the processing unit should be electronically connected to the PT test facilitation device, and more specifically to a digital display of the PT test facilitation device. The PT test facilitation device should be understood to be a computing device comprising either a digital display, an audio output device, or a combination of both. The PT test facilitation device may also comprise a user interface such as a keyboard or touchscreen so that a supervisor may enter, alter, copy, transfer or otherwise manipulate data relevant to a PT test. The PT test facilitation device may conceivably be any computing device which can be used to facilitate the present invention, such as, but not limited to, a desktop computer, a laptop computer, a video gaming console, a mobile device, or another apparatus capable of processing data and running software. The PT test facilitation device may be ruggedized to meet MIL-STD standards for durability and resistance to changing electromagnetic environments. Additionally, a secure database should be utilized to store test subject attributes and other data, PT test scores, and any other relevant information. Alternatively or additionally, the present invention may allow PT test scores and other data to be transferred to a removable media storage device for transport elsewhere.

In the preferred embodiment of the present invention, the wearable sensor holder is an arm wearable apparatus. Alternatively, the wearable sensor holder may be a torso or head wearable apparatus. The specific form of the wearable sensor holder is not limited beyond the capability to be worn by or removably attached to the body of a test subject and being able to support one or more of the subject evaluation sensors adjacent to the test subject's body as desired.

In the preferred embodiment of the present invention, the plurality of subject evaluation sensors comprises an optical sensor, a position sensor, and a movement sensor. The optical sensor is preferably a digital camera or a similar device which can capture images of the test subject, such as, but not limited to, a motion sensor device with gesture control. In alternative embodiments, this optical sensor may be ruggedized to meet MIL-STD standards for durability and resistance to changing electromagnetic environments. The optical sensor may alternatively be an infrared camera, one or more photoelectric sensors, one or more laser sensors, or other optical sensors as useful to the task of capturing environmental data in order to recognize the body of a test subject.

In the preferred embodiment, the position sensor is a global positioning system (GPS) module, though any other position sensors may be utilized as useful and applicable, such as, but not limited to, proximity sensors such as a radio frequency identification (RFID) device. The intent of the position sensor is to ascertain and track the location of a test subject during a run event. The movement sensor is preferably an accelerometer, multiple accelerometers or similar devices which can detect any movements made by the test subject during a PT test. In the preferred embodiment, the position sensor and the movement sensor are connected to the wearable sensor apparatus. Thus, the position sensor and the movement sensor are carried by the test subject and any movement the test subject makes while wearing the wearable sensor apparatus may be tracked by the present invention.

The plurality of subject evaluation sensors is not limited to the aforementioned sensors and may comprise any additional sensors which are useful for evaluating a test subject and the test subject's performance on a PT test. Such additional sensors may include, but are not limited to, a sound sensor, a weight sensor, and a temperature sensor. The sound sensor may be useful for recording sounds such as the test subject confirming their identity or responding to questions, or recording the test subject's breathing rate, exercise repetition rate or other data. Additionally, it is contemplated that in one embodiment the sound sensor or multiple sound sensors may possibly be utilized to more robustly recognize the test subject's body through echolocation if feasible. The weight sensor is utilized to weigh the test subject as part of ascertaining test subject parameters, which are the subject's physical attributes such as height, weight, and other attributes. The weight sensor may be electronically connected to the processing unit through a wireless communication device in order to automatically transmit the test subject's weight to the processing unit. The temperature sensor allows the present invention to monitor the test subject's body temperature as part of the test subject data collected by the present invention. Additional wireless communication devices may be employed in conjunction with various components of the present invention as required where the components are not electronically connected through physical wired connections.

The present invention additionally comprises PT software which is executed through the PT test facilitation device in order to execute a PT test through the PT test facilitation device. The following describes various functions operations and interactions of the PT software.

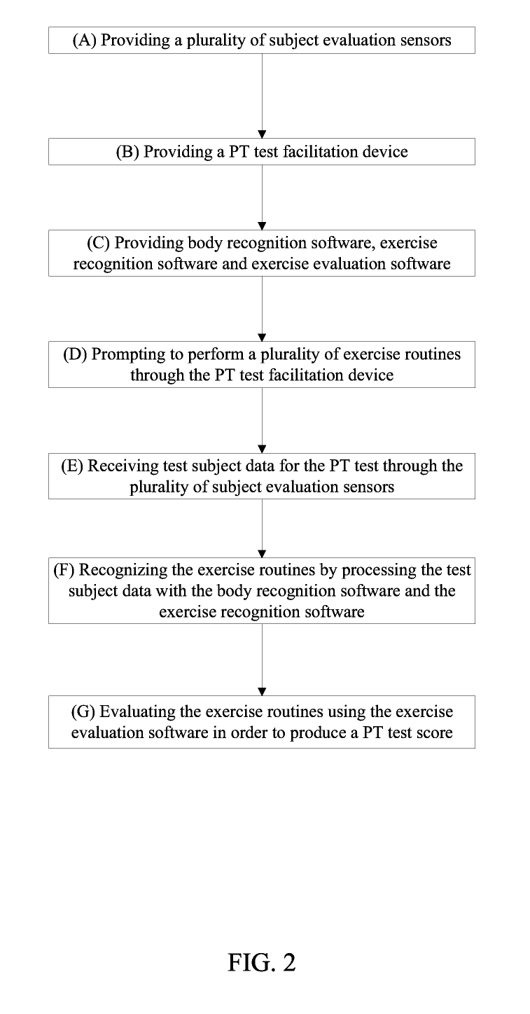

Referring to FIG. 2, in the software method of the present invention, the plurality of subject evaluation sensors are provided, in addition to body recognition software, exercise recognition software, exercise evaluation software, and the PT test facilitation device itself. The PT test facilitation device is not necessarily a component of the present invention, rather a device through which the present invention is facilitated. The body recognition software is utilized primarily in conjunction with the optical sensor in order to recognize the test subject's body and the major parts of the test subject's body such as the head, torso and limbs. The test subject's body must be recognized in order to process and evaluate movements made by the test subject. The body recognition software may also conceivably utilize data gathered from the movement sensor and the sound sensor.

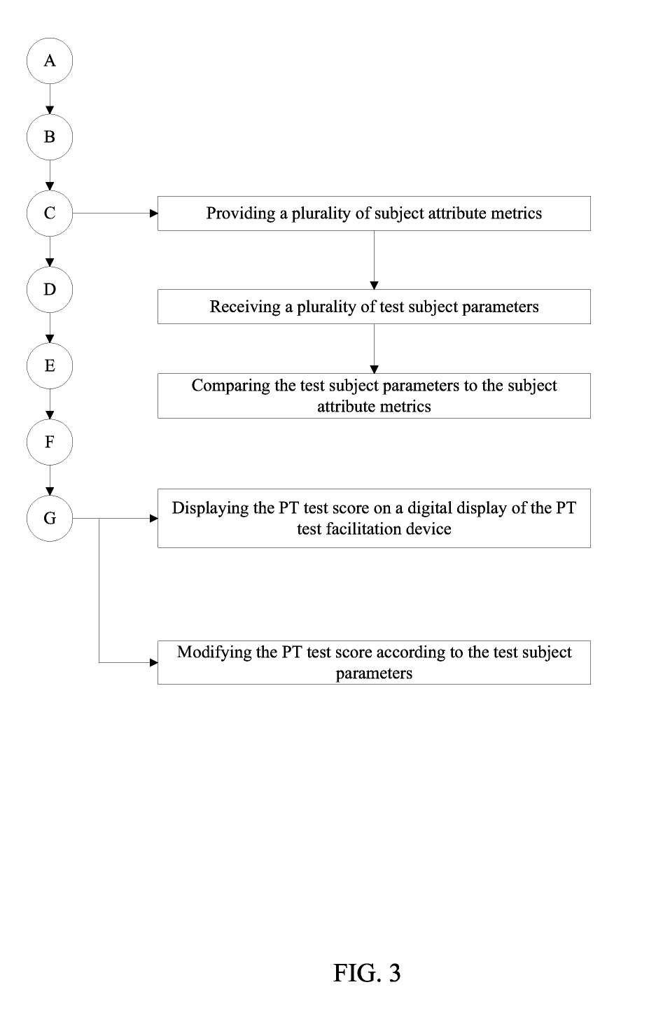

In reference to FIG. 3, a plurality of test subject parameters are gathered and inputted into the system. The test subject parameters are attributes of the test subject such as, but not limited to, the test subject's height, weight, age, body fat composition, resting heart rate, or other attributes which are relevant for evaluating performance on a PT test. The test subject parameters may be received by the processing unit either before or after the PT test is administered. The test subject's height and other bodily dimensions may be acquired through the optical sensor. A plurality of subject attribute metrics are provided, which represent prescriptive regulations for how various test subject parameters may affect the PT test score, in order to control for varying attributes among test subjects. For example, the subject attribute metrics may include a score modifier based on the weight of the test subject. Heavier or lighter test subjects may be required to perform a different number of repetitions in order to achieve the same score, for example. The test subject parameters are compared to the subject attribute metrics, and the PT test score is ultimately modified according to the test subject parameters.

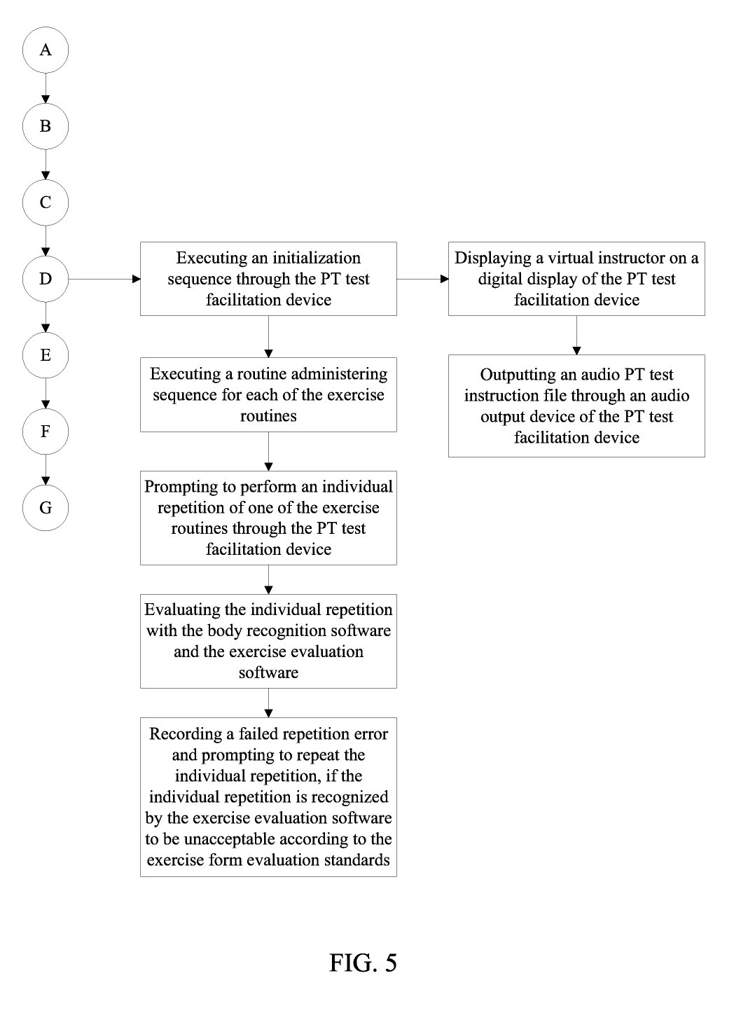

In reference to FIG. 5, the test subject is prompted to perform a plurality of exercise routines through the PT test facilitation device. First, an initialization sequence is executed through the PT test facilitation device. Subsequently, a routine administering sequence is executed for each of the exercise routines through the PT test facilitation device. An exercise routine, or physical training event, is defined as a particular physical activity or set of activities which the test subject performs for assessment. For example, an exercise routine can be a test comprising push-ups, sit-ups, and a two mile run, or an exercise routine can comprise only one of the aforementioned exercises or a different combination of exercises.

To this end, in the preferred embodiment of the present invention, a virtual instructor is utilized to facilitate the PT test. The visual representation of the virtual instructor may be, but is not limited to, a pre-recorded video of a real drill instructor, or an animated and/or computer-generated model which can be programmed to mimic human movements and actions. The virtual instructor is displayed on the digital display of the PT test facilitation device. Additionally, an audio PT test instruction file is outputted through an audio device of the PT test facilitation device, wherein the audio PT test instruction file conveys audible instructions for performing the PT test.

For example, in the preferred embodiment, the virtual instructor greets the test subject and then dictates in a motivating manner the purpose of the test subject's presence as well as rules, regulations, and any other relevant doctrine for the PT test, similar to how a PT test is typically performed by a real instructor. The virtual instructor also executes demonstrations for each of the exercise routines in order to specify what is correct and what will not be counted. Subsequently, the virtual instructor dictates administrative data such as, but not limited to, the test subject's social security number or other identification information in order to confirm the test subject's identity.

During the PT test, test subject data is received through the plurality of subject evaluation sensors. The test subject data refers to any data collected by any of the plurality of test subject evaluation sensors. In the preferred embodiment, the test subject should approach a designated marking on the floor so which is designated in order to properly calibrate the optical sensor. The optical sensor captures images of the test subject in order to assess the test subject's physical measurements. In the preferred embodiment physical markers on the floor indicate proper positions for the test subject to place their hands and/or feet while performing the exercise routines.

The exercise routines performed by the test subject are evaluated by processing the test subject data with the body recognition software and the exercise recognition software. In the preferred embodiment of the present invention, the images captured by the optical sensor (camera) are displayed live on the digital display for the test subject as well as any supervisor or other personnel in the vicinity to observe while the test subject is performing the exercise routines.

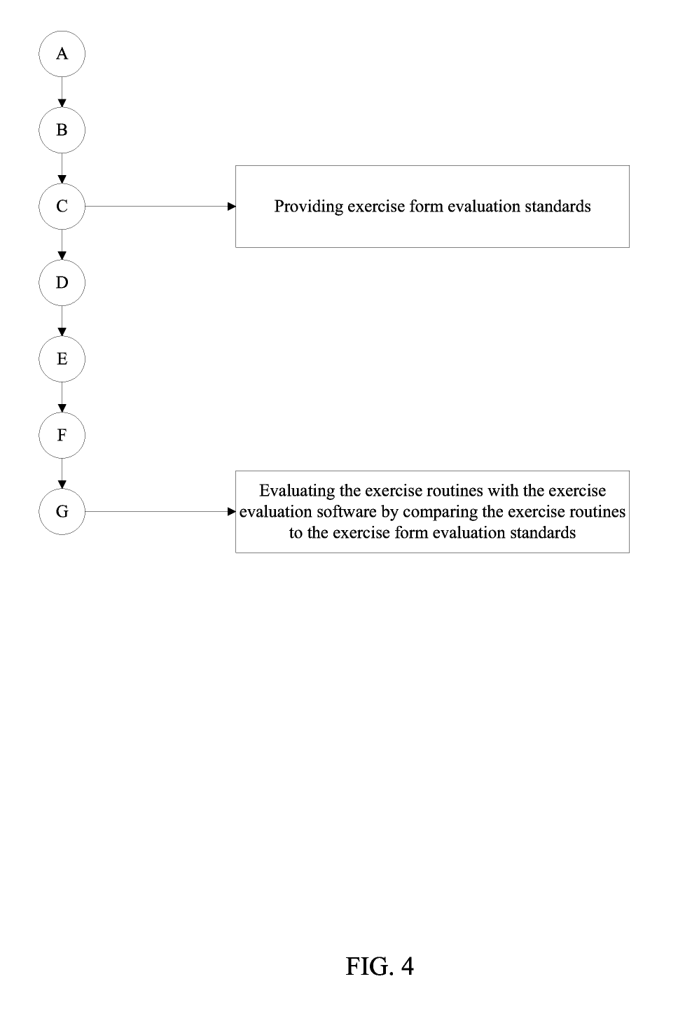

Exercise form evaluation standards are provided with the exercise evaluation software, as shown in FIG. 4. The exercise form evaluation standards are preferably an instruction file or files which provide metrics for recognizing an acceptable exercise to the exercise evaluation software. For example, one such metric may be that on a push-up exercise, the test subject's chest must come within two inches of the ground, or the tricep of the test subject must become parallel to the ground in order for the push-up to be counted as acceptable. The exercise routines are evaluated with the exercise evaluation software by comparing the recognized exercise routines performed by the test subject to the exercise form evaluation standards. For exercise routines which comprise a plurality of repetitions, the test subject is repeatedly prompted to perform an individual repetition of the exercise routines through the PT test facilitation device.

The recognized exercise routines are evaluated using the exercise evaluation software in order to produce a PT test score. More specifically, each individual repetition is evaluated with the body recognition software and the exercise evaluation software. The test subject's body is recognized with the body recognition software, and the exercise evaluation software ascertains if the movements made by the test subject's body conform to the exercise form standards. If the individual repetition is recognized by the exercise evaluation software to be unacceptable according to the exercise form standards, a failed repetition error is recorded and the test subject is prompted to repeat the individual repetition. Furthermore, the virtual instructor will notify the test subject of failed repetitions and the PT test facilitation device will output an audio file to motivate and instruct the test subject to correct their form within regulation.

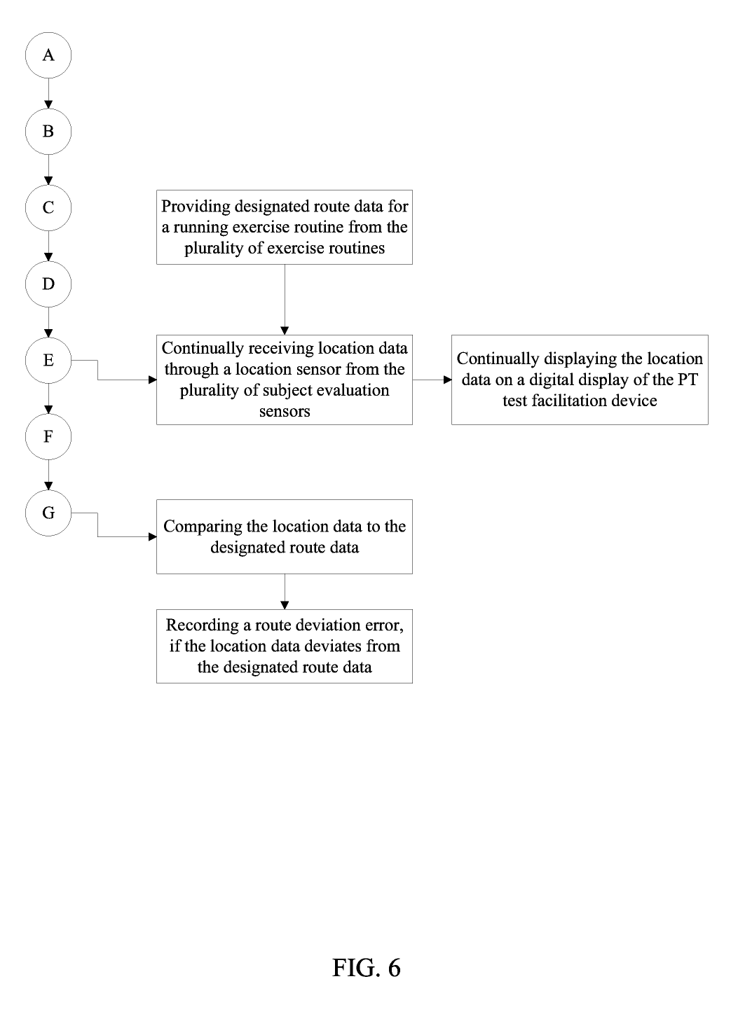

Referring to FIG. 6, one specific exercise routine to be evaluated by the present invention is a running exercise routine from the plurality of exercise routines. The virtual instructor will demonstrate on the digital display a designated route for the running exercise routine. Designated route data is provided to the system for the running exercise routine. While the running exercise routine is underway, location data is continually received from the location sensor, which is connected to the wearable sensor apparatus worn by the test subject while performing the exercise running routine. The location data is compared to the designated route data in order to ensure that the test subject adheres to the designated route. If the received location data deviates from the designated route data, a route deviation error is recorded in the system. In the preferred embodiment, during the running exercise routine, the location data is continually displayed on the digital display so that the PT test administrator can monitor the test subject's progress along the designated route.

Once the PT test is completed, the PT test score is calculated and is displayed on the digital display of the PT test facilitation device.

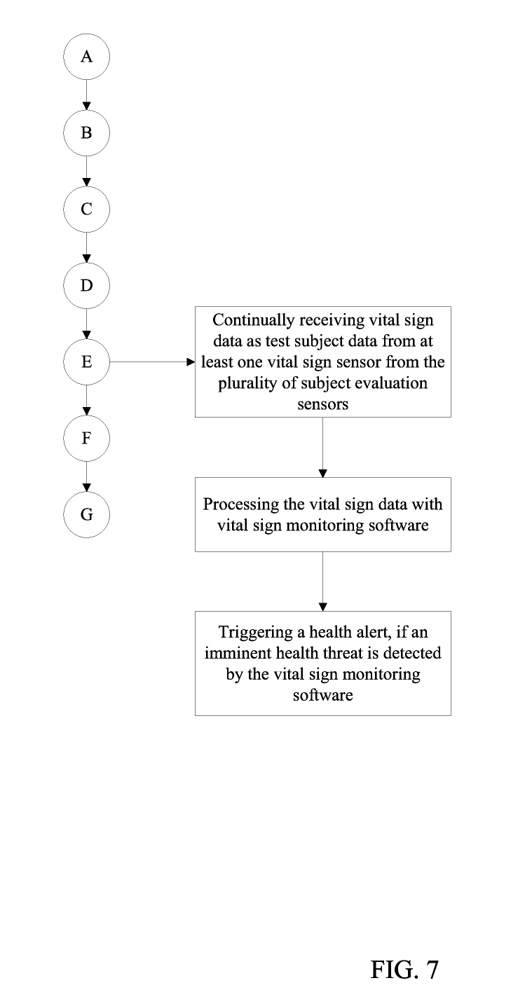

Referring to FIG. 7, additionally, during the PT test vital sign data is continually received as test subject data from at least one vital sign sensor from the plurality of subject evaluation sensors. In the preferred embodiment of the present invention, the vital sign sensors track the test subject's heart rate, body temperature, blood pressure and energy expenditure in real-time. The vital sign data is processed with vital sign monitoring software and a health alert is triggered if an imminent health threat is detected by the vital sign monitoring software. In the preferred embodiment, the vital sign sensors provide the ability to detect imminent health threats such as, but not limited to, heat stroke, hypothermia, drops in blood pressure, and elevated body temperature.

The present invention will grade each test subject fairly and accurately. Software will need to be revised every year to comply with updated regulation, methods, techniques and even graphics.

The aforementioned example is one conceivable embodiment of the present invention. The present invention may include many different scenarios, dependent on the specifics of the PT test and variations in the programming of the software. The invention's software program may include possible add-ons for particular scenarios and is not constrained by the aforementioned scenario's description.

In the preferred embodiment, the secure database is a standard storage repository utilizing a system of servers with multiple layers of encryption. This database is connected to the PT test facilitation device, which uploads the data from each PT test to this database for review. The database will meet industry-standard requirements for network security and PII (personally identifiable information). Such standards may include and are not limited to: NIST 800-53, Health Insurance Portability and Accountability Act (HIPPA), Sarbanes-Oxley requirements (SOX), Payment Card Industry Data Security Standard (PCI DSS), and Federal Information Security Management Act (FISMA).

Future or alternative embodiments of the invention may have additional capabilities, such as the ability to measure body fat composition or track and grade other events. Additionally, future or alternative embodiments of the invention may support data transfer to a USB drive, so the user may take the data home to their personal computing device, such as, but not limited to a gaming console, and continue a physical training regimen there utilizing the present invention.

Although the invention has been explained in relation to its preferred embodiment, it is to be understood that many other possible modifications and variations can be made without departing from the spirit and scope of the invention as hereinafter claimed.

What is claimed is:

1. A physical training test facilitating system comprises:
a wearable sensor holder;
a plurality of subject evaluation sensors;
a processing unit;
the plurality of subject evaluation sensors comprises an optical sensor, a position sensor, and a movement sensor;
the position sensor and the movement sensor being connected to the wearable sensor apparatus;
each of the plurality of subject evaluation sensors being electronically connected to the processing unit, wherein the processing unit receives inputs from the plurality of subject evaluation sensors in order to facilitate a physical training (PT) test using a PT test facilitation device;

2. The physical training test facilitating system as claimed in claim 1 comprises:
the plurality of subject evaluation sensors further comprises a sound sensor;

3. The physical training test facilitating system as claimed in claim 1 comprises:
the plurality of subject evaluation sensors further comprises a weight sensor;

4. The physical training test facilitating system as claimed in claim 1 comprises:
the plurality of subject evaluation sensors further comprises a temperature sensor;
the temperature sensor being connected to the wearable sensor apparatus, wherein the temperature sensor is positioned adjacent to a user's skin while in use and wherein the temperature sensor measures the user's body temperature;

5. The physical training test facilitating system as claimed in claim 1 comprises:
the optical sensor being a camera;

6. The physical training test facilitating system as claimed in claim 1 comprises:
the position sensor being a global positioning system module;

7. The physical training test facilitating system as claimed in claim 1 comprises:
the processing unit being electronically connected to a digital display of the PT test facilitation device;

8. The physical training test facilitating system as claimed in claim 1 comprises:
the wearable sensor holder being an arm wearable apparatus;

9. The physical training test facilitating system as claimed in claim 1 comprises:
the wearable sensor holder being a torso wearable apparatus;

10. A method of using a physical training test facilitating system by executing computer-executable instructions stored on a non-transitory computer-readable medium comprises the steps of:
providing a plurality of subject evaluation sensors;
providing a PT test facilitation device;
providing body recognition software, exercise recognition software and exercise evaluation software;
prompting to perform a plurality of exercise routines through the PT test facilitation device;
receiving test subject data for the PT test through the plurality of subject evaluation sensors;
recognizing the exercise routines by processing the test subject data with the body recognition software and the exercise recognition software;
evaluating the exercise routines using the exercise evaluation software in order to produce a PT test score;

11. A method of using a physical training test facilitating system by executing computer-executable instructions stored on a non-transitory computer-readable medium as claimed in claim 10 comprises the step of:
displaying the PT test score on a digital display of the PT test facilitation device;

12. A method of using a physical training test facilitating system by executing computer-executable instructions stored on a non-transitory computer-readable medium as claimed in claim 10 comprises the steps of:
providing a plurality of subject attribute metrics;
receiving a plurality of test subject parameters;
comparing the test subject parameters to the subject attribute metrics;
modifying the PT test score according to the test subject parameters;

13. A method of using a physical training test facilitating system by executing computer-executable instructions stored on a non-transitory computer-readable medium as claimed in claim 10 comprises the steps of:
providing exercise form evaluation standards;
evaluating the exercise routines with the exercise evaluation software by comparing the exercise routines to the exercise form evaluation standards;

14. A method of using a physical training test facilitating system by executing computer-executable instructions stored on a non-transitory computer-readable medium as claimed in claim 10 comprises the steps of:
executing an initialization sequence through the PT test facilitation device;
executing a routine administering sequence for each of the exercise routines;

15. A method of using a physical training test facilitating system by executing computer-executable instructions stored on a non-transitory computer-readable medium as claimed in claim 14 comprises the steps of:
displaying a virtual instructor on a digital display of the PT test facilitation device;
outputting an audio PT test instruction file through an audio output device of the PT test facilitation device, wherein the audio PT test instruction file conveys audible instructions for performing a PT test;

16. A method of using a physical training test facilitating system by executing computer-executable instructions stored on a non-transitory computer-readable medium as claimed in claim 14 comprises the steps of:
prompting to perform an individual repetition of one of the exercise routines through the PT test facilitation device;
evaluating the individual repetition with the body recognition software and the exercise evaluation software;
recording a failed repetition error and prompting to repeat the individual repetition, if the individual repetition is recognized by the exercise evaluation software to be unacceptable according to the exercise form evaluation standards;

17. A method of using a physical training test facilitating system by executing computer-executable instructions stored on a non-transitory computer-readable medium as claimed in claim 10 comprises the steps of:
providing designated route data for a running exercise routine from the plurality of exercise routines;
continually receiving location data through a location sensor from the plurality of subject evaluation sensors;
comparing the location data to the designated route data;
recording a route deviation error, if the location data deviates from the designated route data;

18. A method of using a physical training test facilitating system by executing computer-executable instructions stored on a non-transitory computer-readable medium as claimed in claim 17 comprises the steps of:
continually displaying the location data on a digital display of the PT test facilitation device;

19. A method of using a physical training test facilitating system by executing computer-executable instructions stored on a non-transitory computer-readable medium as claimed in claim 10 comprises the steps of:
continually receiving vital sign data as test subject data from at least one vital sign sensor from the plurality of subject evaluation sensors;
processing the vital sign data with vital sign monitoring software;
triggering a health alert, if an imminent health threat is detected by the vital sign monitoring software;
